(19) United States
(12) Patent Application Publication
(10) Pub. No.: US 2021/0109080 A1
(43) Pub. Date: Apr. 15, 2021

(54) Cell-Based Biosensor for Early Alzheimer's Disease Detection

(71) Applicant: University of South Carolina, Columbia, SC (US)

(72) Inventors: Melissa A. Moss, Columbia, SC (US); Brittany E. Watson, McComb, MS (US)

(73) Assignee: University of South Carolina, Columbia, SC (US)

(21) Appl. No.: 17/071,020

(22) Filed: Oct. 15, 2020

Related U.S. Application Data
(60) Provisional application No. 62/915,208, filed on Oct. 15, 2019.

(51) Int. Cl. G01N 33/483; G01N 33/487

(52) U.S. Cl. CPC G01N 33/4836; G01N 2800/2821; G01N 33/48785

(57) ABSTRACT

The present disclosure has identified a unique cellular response to the pathogenic form of the amyloid-ß protein (Aß) and will employ a cell-based biosensor to leverage this response for early detection of Alzheimer's disease (AD) by determining if the pathogenic form of Aß is present in the cerebral spinal fluid or blood of a patient.

### BACKGROUND

### 1) Technical Field

The subject matter disclosed herein is generally directed to a unique cellular response to the pathogenic form of the amyloid-ß protein (Aß) and will employ a cell-based biosensor to leverage this response for early diagnosis of Alzheimer's disease (AD) by determining if the pathogenic form of Aß is present in the cerebral spinal fluid or blood of a patient.

### 2) Background

AD, a neurodegenerative disease characterized by progressive cognitive decline, is a leading cause of dementia in people over the age of sixty. Due to the irreversible death of neurons, early diagnosis of the disease is crucial for effective treatment. AD is the 6th leading cause of death in the United States (US), and 5.8 million Americans are currently living with AD. Between the years 2000 and 2018, deaths from heart disease have decreased by 7.8%, while the deaths from AD have increased 146%. By the year 2050, the number of people aged sixty-five and older with AD is projected to reach 13.8 million in the US alone, thus the potential market is large. The 2020 Alzheimer's Facts and Figures Report states family or unpaid caregivers provide 18.6 billion hours of care, which is valued over $244 billion per year. If an early diagnostic tool became available, $7 trillion is the estimated potential cost savings for the current US population.

AD is characterized by deposition of aggregated Aß in the brain, and thus Aß has been proposed as a disease biomarker. Early detection of this biomarker has proven ineffective with traditional biochemical measurements due to the challenge of detecting small quantities of physiologically active Aß aggregates in the presence of much larger quantities of inactive Aß monomer. A cost-effective method for early diagnosis does not exist. Magnetic resonance imaging (MRI) can detect a decrease in the size of different regions of the brain. AD mainly affects the temporal and parietal lobes, but changes in size occur only at late stages of the disease. Positron emission tomography (PET) scan can reveal abnormal protein clusters in the brain, which also occur only at more advanced stages of the disease.

Accumulation of amyloid plaques comprised of aggregated Aß characterizes AD. Currently, there are blood tests in development, but not on the market, which measure changes in the quantity of total Aß present in the blood; however, these tests do not specifically detect pathogenic forms of Aß nor employ cellular responses as disclosed herein. While monomeric Aß is inert, oligomeric aggregates of the protein, which appear early in disease progression, have been identified as the most pathogenic species. Thus, detection of these Aß oligomers can provide early diagnosis. Accordingly, it is an object of the present disclosure to provide a cell-based biosensor and methods for early diagnosis of AD. Citation or identification of any document in this application is not an admission that such a document is available as prior art to the present disclosure.

### SUMMARY

The above objectives are accomplished according to the present disclosure by providing in one embodiment, a method for an early diagnostic method of Alzheimer's Disease. The method may include measuring transendothelial electrical resistance in a monolayer of brain microvascular endothelial cells, introducing a fluid sample to the monolayer of brain microvascular endothelial cells, and determining if introduction of the fluid sample causes a change in the transendothelial electrical resistance of the monolayer of brain microvascular endothelial cells, wherein a decrease in transendothelial electrical resistance is indicative of Alzheimer's disease. Further, the decrease in transendothelial electrical resistance may be indicative of the presence of at least one pathogenic Aß aggregate. Yet still, a synthetic monomer may be used to amplify a concentration of at least one physiologically active Aß aggregate to increase compromise of the monolayer of brain microvascular endothelial cells. Further, no amplification may signify absence of at least one pathogenic Aß aggregate. Moreover, the fluid samples may include at least one cerebral spinal fluid sample and/or at least one blood sample. Still, the method may detect Aß aggregation including Aß oligomerization. Still yet, the method may include measuring transendothelial transfer rate of the monolayer of brain microvascular endothelial cells. Furthermore, the brain microvascular endothelial cells may comprise human brain microvascular endothelial cells. Yet again, no change in resistance occurs when at least one Aß monomer or at least one mature Aß fibril is present.

In a further embodiment, the current disclosure provides for using a cell-based biosensor to diagnose Alzheimer's Disease. The method may include forming a monolayer of brain microvascular endothelial cells, exposing the monolayer of brain microvascular endothelial cells to at least one fluid sample, and determining the presence of at least one pathogenic Aß aggregate in the at least one fluid sample via a change in electrical resistance within the monolayer of brain microvascular endothelial cells, wherein no change in electrical resistance signifies an absence of the at least one pathogenic Aß aggregate. Further, a synthetic monomer may be used to amplify a concentration of at least one physiologically active Aß aggregate to increase compromise of the monolayer of brain microvascular endothelial cells. Still again, no amplification may signify an absence of the at least one pathogenic Aß aggregate. Yet still, the fluid samples may include at least one cerebral spinal fluid sample and/or at least one blood sample. Again, the method may detect Aß aggregation including Aß oligomerization. Still again, the method may include measuring transendothelial transfer rate of the monolayer of brain microvascular endothelial cells. Furthermore, the brain microvascular endothelial cells may comprise human brain microvascular endothelial cells. Again yet, no change in resistance occurs when at least one Aß monomer or at least one mature Aß fibril is present.

These and other aspects, objects, features, and advantages of the example embodiments will become apparent to those having ordinary skill in the art upon consideration of the following detailed description of example embodiments.

### BRIEF DESCRIPTION OF THE DRAWINGS

The construction designed to carry out the disclosure will hereinafter be described, together with other features thereof. The invention will be more readily understood from a reading of the following specification and by reference to the accompanying drawings forming a part thereof, wherein an example of the disclosure is shown and wherein:

FIG. 1 shows a simplified illustration of the blood-brain barrier (BBB).

FIG. 2 shows an illustration of Aß aggregation, including early oligomerization.

FIG. 3 shows Aß aggregates increase endothelial permeability via (a) increased transendothelial transfer rate of a large protein molecule and (b) decreased transendothelial electrical resistance (TEER).

FIG. 4 shows small Aß aggregates selectively (a) increase the transendothelial transfer of a large protein molecule, expressed as the permeability coefficient (Pₑ), and (b) decrease TEER. In contrast, Aß monomer and Aß fibril fail to induce this response.

FIG. 5 shows increased endothelial Pₑ correlates inversely with Aß aggregate size.

FIG. 6 shows small Aß aggregates selectively induce relocalization of tight junction-associated protein ZO-1. In contrast, Aß monomer and Aß fibril fail to induce this response.

FIG. 7 shows (a) transcription factor NF-κB is activated following Aß aggregate treatment at concentrations of Aß aggregates that parallel physiological levels, and (b) NF-κB is involved in the Aß aggregate-induced changes in permeability.

FIG. 8 shows cultivation of human brain microvascular endothelial cells (HBMVECs) followed by treatment with Aß oligomers to yield the measurable endothelial permeability response.

FIG. 9 shows TEER is reduced following Aß oligomer treatment.

FIG. 10 shows amplification of low Aß oligomer concentrations.

It will be understood by those skilled in the art that one or more aspects of this disclosure can meet certain objectives, while one or more other aspects can meet certain other objectives. Each objective may not apply equally, in all its respects, to every aspect of this disclosure. As such, the preceding objects can be viewed in the alternative with respect to any one aspect of this disclosure. These and other objects and features of the disclosure will become more fully apparent when the following detailed description is read in conjunction with the accompanying figures and examples. However, it is to be understood that both the foregoing summary of the disclosure and the following detailed description are of a preferred embodiment and not restrictive of the disclosure or other alternate embodiments of the disclosure. In particular, while the disclosure is described herein with reference to a number of specific embodiments, it will be appreciated that the description is illustrative of the disclosure and is not constructed as limiting of the disclosure. Various modifications and applications may occur to those who are skilled in the art, without departing from the spirit and the scope of the disclosure, as described by the appended claims. Likewise, other objects, features, benefits and advantages of the present disclosure will be apparent from this summary and certain embodiments described below, and will be readily apparent to those skilled in the art. Such objects, features, benefits and advantages will be apparent from the above in conjunction with the accompanying examples, data, figures and all reasonable inferences to be drawn therefrom, alone or with consideration of the references incorporated herein. The figures herein are for illustrative purposes only and are not necessarily drawn to scale.

### DETAILED DESCRIPTION OF A PREFERRED EMBODIMENT

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further embodiment includes from the one particular value and/or to the other particular value. The recitation of numerical ranges by endpoints includes all numbers and fractions subsumed within the respective ranges, as well as the recited endpoints. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R.I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosure. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, a “biological sample” may contain whole cells and/or live cells and/or cell debris. The biological sample may contain (or be derived from) a “bodily fluid”. The present disclosure encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, and cell cultures from bodily fluids. Bodily fluids may be obtained from a mammal organism, for example by puncture, or other collecting or sampling procedures.

As used herein, “agent” refers to any substance, compound, molecule, and the like, which can be administered to a subject on a subject to which it is administered to. An agent can be inert. An agent can be an active agent. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

As used herein, “active agent” or “active ingredient” refers to a substance, compound, or molecule, which is biologically active or otherwise induces a biological or physiological effect on a subject to which it is administered to. In other words, “active agent” or “active ingredient” refers to a component or components of a composition to which the whole or part of the effect of the composition is attributed.

As used herein, “administering” refers to any suitable administration for the agent(s) being delivered and/or subject receiving said agent(s) and can be oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition to the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated, subject being treated, and/or agent(s) being administered. The current disclosure has identified a unique cellular response to the pathogenic form of Aß. A cell-based biosensor will leverage this response for detection of the disease by determining if the pathogenic form of Aß is present in the cerebral spinal fluid or blood of a patient.

The term “molecular weight”, as used herein, can generally refer to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (Mw) as opposed to the number-average molecular weight (Mn). Capillary viscometry provides estimates of molecular weight as the inherent viscosity determined from a dilute polymer solution using a particular set of concentration, temperature, and solvent conditions.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used interchangeably herein, the terms “sufficient” and “effective,” can refer to an amount (e.g. mass, volume, dosage, concentration, and/or time period) needed to achieve one or more desired and/or stated result(s). For example, a therapeutically effective amount refers to an amount needed to achieve one or more therapeutic effects.

As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g. a web interface.

As used herein, “therapeutic” can refer to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as cancer and/or indirect radiation damage. The effect can be therapeutic in terms of a partial or complete cure of a disease, condition, symptom or adverse effect attributed to the disease, disorder, or condition. The term “treatment” as used herein covers any treatment of, Alzeimer's or similar conditions, in a subject, particularly a human and/or companion animal, and can include any one or more of the following: (a) preventing the disease or damage from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

As used herein, the terms “weight percent,” “wt %,” and “wt. %,” which can be used interchangeably, indicate the percent by weight of a given component based on the total weight of a composition of which it is a component, unless otherwise specified. That is, unless otherwise specified, all wt % values are based on the total weight of the composition. It should be understood that the sum of wt % values for all components in a disclosed composition or formulation are equal to 100. Alternatively, if the wt % value is based on the total weight of a subset of components in a composition, it should be understood that the sum of wt % values the specified components in the disclosed composition or formulation are equal to 100.

As used herein, “water-soluble” generally means at least about 10 g of a substance is soluble in 1 L of water, i.e., at neutral pH, at 25° C.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present disclosure. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure. For example, in the appended claims, any of the claimed embodiments can be used in any combination.

All patents, patent applications, published applications, and publications, databases, websites and other published materials cited herein are hereby incorporated by reference to the same extent as though each individual publication, published patent document, or patent application was specifically and individually indicated as being incorporated by reference.

Kits

Any of the compounds and/or formulations described herein can be presented as a combination kit. As used herein, the terms “combination kit” or “kit of parts” refers to the compounds, compositions, formulations, particles, cells and any additional components that are used to package, sell, market, deliver, and/or administer the combination of elements or a single element, such as the active ingredient, contained therein. Such additional components include, but are not limited to, packaging, syringes, blister packages, bottles, and the like. When one or more of the compounds, compositions, formulations, particles, cells, described herein or a combination thereof (e.g., agent(s)) contained in the kit are administered simultaneously, the combination kit can contain the active agent(s) in a single formulation, such as a pharmaceutical formulation, (e.g., a tablet, liquid preparation, dehydrated preparation, etc.) or in separate formulations. When the compounds, compositions, formulations, particles, and cells described herein or a combination thereof and/or kit components are not administered simultaneously, the combination kit can contain each agent or other component in separate pharmaceutical formulations. The separate kit components can be contained in a single package or in separate packages within the kit.

In some embodiments, the combination kit also includes instructions printed on or otherwise contained in a tangible medium of expression. The instructions can provide information regarding the content of the compounds and/or formulations, safety information regarding the content of the compounds and formulations (e.g., pharmaceutical formulations), information regarding the dosages, indications for use, and/or recommended treatment regimen(s) for the compound(s) and/or pharmaceutical formulations contained therein. In some embodiments, the instructions can provide directions and protocols for administering the compounds and/or formulations described herein to a subject in need thereof. In some embodiments, the instructions can provide one or more embodiments of the methods for administration and/or pharmaceutical formulation thereof such as any of the methods described in greater detail elsewhere herein.

Accumulation of amyloid plaques comprised of aggregated Aß, neuronal death, and BBB breakdown characterize AD. The BBB is formed by a tight monolayer of endothelial cells, which is characterized by tight junctions, the absence of openings, and few pinocytotic vesicles. Tight junctions are required for a healthy, intact monolayer and render relatively high TEER. As permeability of the monolayer increases, the TEER values decrease. AD-associated breakdown of the BBB is correlated with vascular Aß deposition, suggesting that pathological Aß can modulate TEER. This research considers how this physiological response might be leveraged for early disease diagnosis.

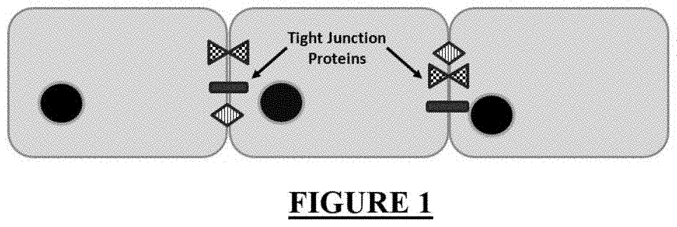

FIG. 1 shows a simplified illustration of the BBB. A single endothelial cell thickness comprises the capillary wall in the brain. Tight junction proteins work to ensure selectivity of molecules entering the brain. The current disclosure focuses on the negative effects of Aß oligomers to the BBB.

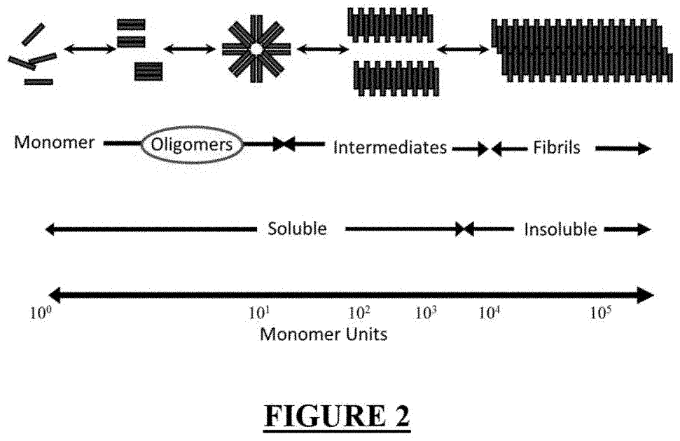

FIG. 2 shows the pathway for Aß aggregation. In healthy brains, Aß is present in its monomeric form. However, Aß monomer can undergo aggregation, forming oligomers, soluble intermediates, and ultimately fibrils which deposit in the brain as plaques.

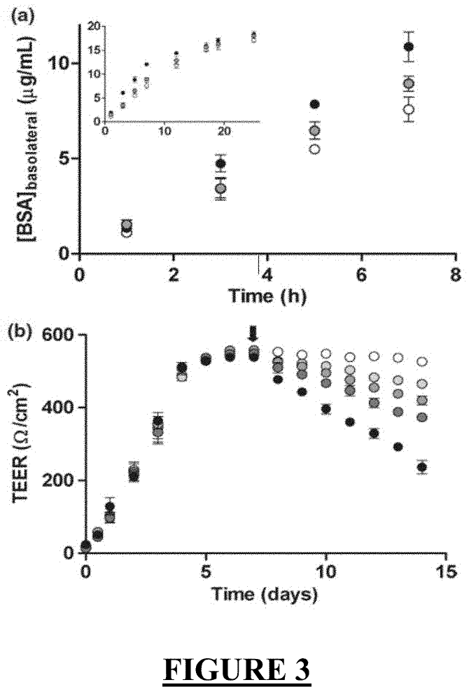

FIG. 3 shows Aß aggregates increase endothelial permeability. HBMVEC monolayers were incubated alone (white circles), with 0.3-10 μM Aß aggregates (grey circles), or with TNF-α (positive control, black circles). (a) Following 24-h treatment, transfer of FITC-BSA from the apical to basolateral chamber was monitored via florescence. Error bars represent SEM, n=3. Increased permeability is evidenced by a faster transfer rate, or steeper slope. Following treatment with 5 μM Aß aggregates, the transfer rate is increased by approximately 10%. (b) TEER was monitored following addition of treatments (indicated by arrow). Increased permeability is evidenced by decreased TEER. In the absence of Aß aggregates, TEER maintains a plateau, decreasing by less than 8% over 7 days. In the presence of 10 μM Aß aggregates, TEER exhibits a dose-dependent decrease of approximately 35% over 7 days.

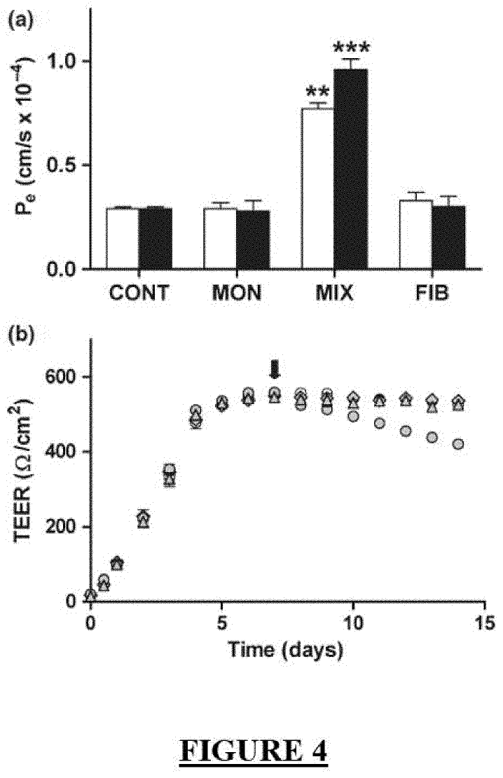

FIG. 4 shows the Aß aggregate-induced increase endothelial permeability is selective for small aggregates. HBMVEC monolayers were incubated alone (CONT, white circles), with Aß monomer (MON, diamonds), with Aß aggregate mixture containing small aggregates (MIX, circles), or with Aß fibrils (FIB, triangles). (a) Using FITC-BSA transfer rates, Pₑ was determined after 24-h treatment with Aß concentrations of 5 μM (white bars) and 10 μM (black bars). Error bars represent SEM, n=3. **p<0.01 Increased permeability, evidenced by increased Pₑ, is observed only for treatment with Aß aggregate mixture containing small aggregates. (b) TEER was monitored following addition of treatments at a concentration of 5 μM (indicated by arrow). Increased permeability, evidenced by decreased TEER, is observed only for treatment with Aß aggregate mixture containing small aggregates.

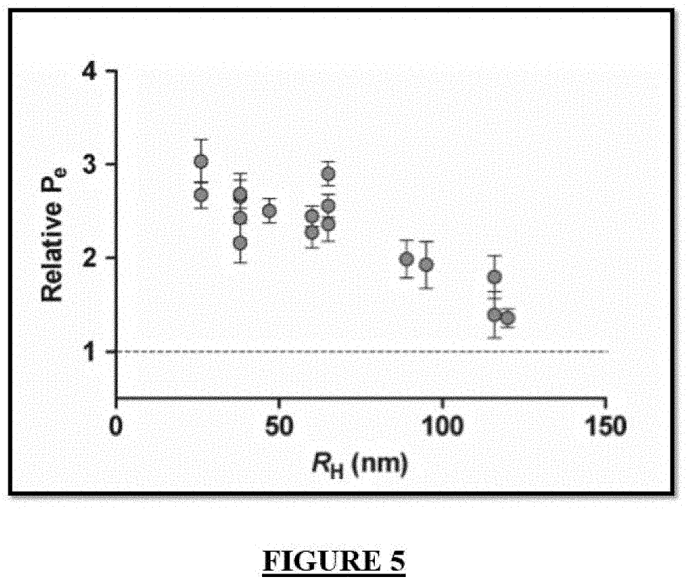

FIG. 5 shows increased endothelial Pₑ correlates inversely with Aß aggregate size. HBMVEC monolayers were incubated (24 h) alone or with 5 μM Aß aggregates exhibiting a range of average hydrodynamic radii (RH). Pₑ is determined from the transfer rate of FITC-BSA and reported relative to untreated monolayers. Error bars represent SEM, n=3. Increased permeability, evidenced by increased relative Pₑ, is more pronounced for smaller aggregates.

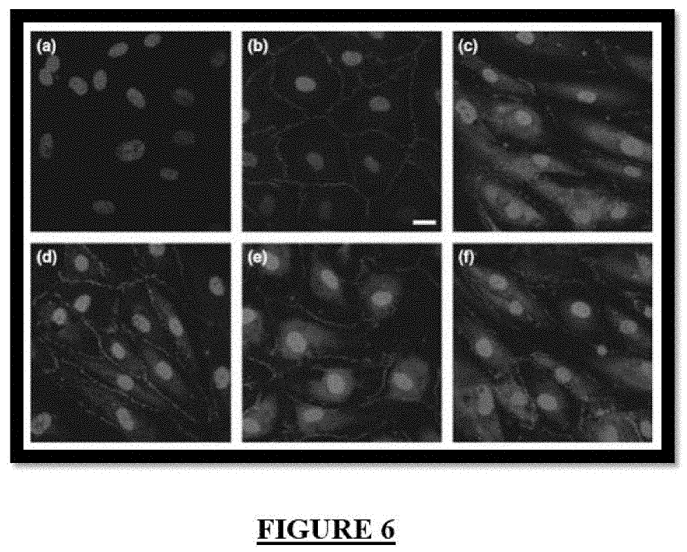

FIG. 6 shows small Aß aggregates induce breakdown of BBB tight junctions. Confluent HBMVEC monolayers were incubated (24 h) alone (b), with TNF-α (positive control, c), or with 2.5, 5.0, or 10 μM Aß aggregates (d, e, and f, respectively). Immunofluorescence staining was performed for tight junction-associated protein ZO-1 (red) in conjunction with nuclear (DAPI, blue) staining. Negligible background was observed in the absence of ZO-1 primary antibody (a). Images shown are representative of ten separate images acquired per sample. Images are shown relative to a scale bar of 20 μm. Relocalization of ZO-1 away from cell borders and into the cytoplasm indicates breakdown of BBB tight junctions.

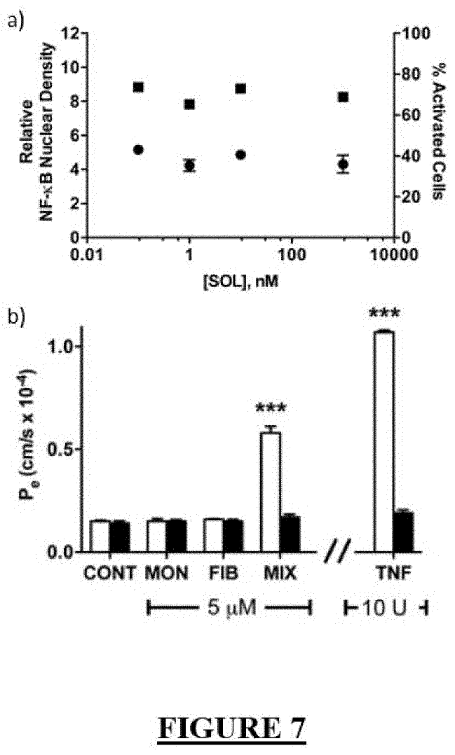

FIG. 7 shows transcription factor NF-κB is activated following Aß aggregate treatment and responsible for Aß aggregate-induced changes in permeability. (a) Confluent HBMVEC monolayers were incubated for 30 min with 100 pM, 1 nM, 10 nM, or 1 μM isolated small soluble Aß aggregates (SOL) (R_{H}=43-54 nm). NF-κB nuclear localization, indicative of NF-κB activation, was assessed via immunocytochemistry and quantitative image analysis. Results are reported as NF-κB nuclear density relative to the untreated control (circles) and the percentage of cells containing activated NF-κB (squares). Error bars represent SEM, n=3. Concentrations of Aß aggregates ranging from 1 μM and down to 10 pM activate similar levels of cell signaling. (b) Confluent HBMVEC monolayers were pre-treated for 30 min alone (white bars) or with 3 μM MG-132, an inhibitor of NF-κB (black bars). Monolayers were then treated (24 h) alone (CONT), with TNF-α (positive control, TNF), with 5 μM Aß monomer (MON), with 5 μM Aß fibrils (FIB), or with 5 μM Aß aggregate mixture containing small aggregates (MIX). Using FITC-BSA transfer rates, Pₑ was determined. Error bars represent SEM, n=3. ***p<0.001 Abrogation of the Aß-induced increase in Pₑ by the presence of MG-132 demonstrates the involvement of NF-κB in the signaling pathway.

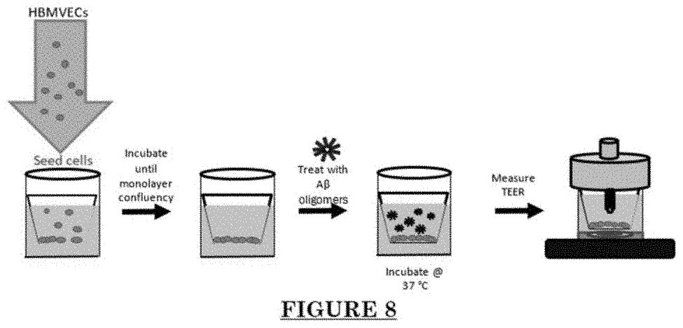

FIG. 8 shows HBMVEC monolayers were cultured on a suspended membrane to mimic the BBB. Cell culture media was supplemented with 550 nM hydrocortisone to promote barrier properties. Once monolayers reached confluence, they were treated with Aß oligomers. TEER was measured using EndOhm and EVOM2.

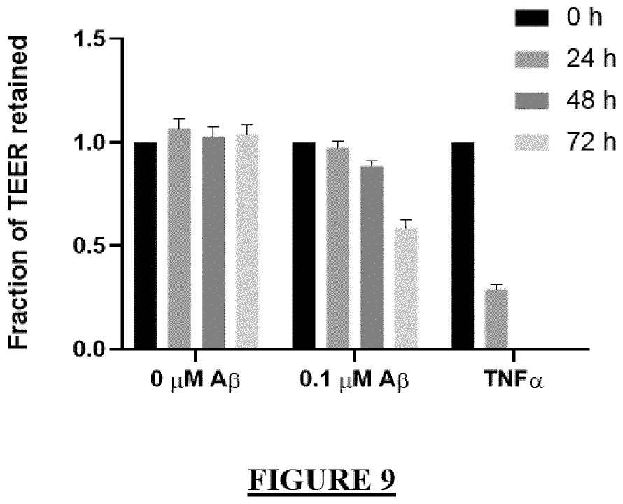

FIG. 9 shows TEER is reduced following Aß oligomer treatment. HBMVECs were grown to confluence until TEER values reached a plateau. Confluent HBMVEC monolayers were treated with at Aß oligomers at concentrations of 0 μM (vehicle) or 0.1 μM, or with TNF-α (positive control). TEER was measured 24, 48, and 72 h after treatment, and values are reported at the fraction of TEER observed prior to treatment. Error bars represent SEM, n=3-7. TEER remains steady in the presence of vehicle and reduces following 72 h treatment with 0.1 μM Aß oligomers.

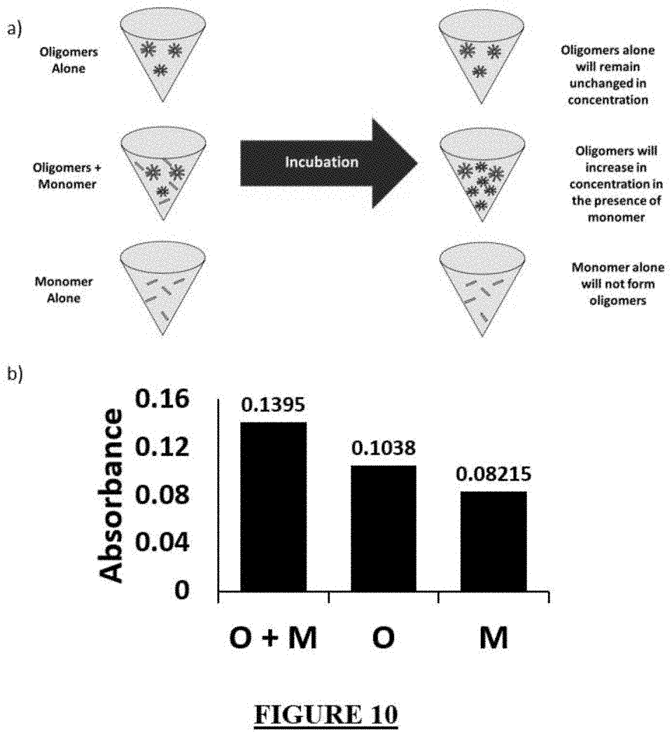

FIG. 10 shows amplification of low Aß oligomer concentrations. (a) Low concentrations of physiologically active Aß oligomers can be amplified via addition of synthetic Aß monomer including Aß₁₋₄₀, Aß₁₋₄₂, and any mutants of shorter isoforms of Aß to increase compromise of endothelial monolayers and induce a larger change in TEER. These characteristics will allow selective amplification of signal from patient-derived samples contacting pathogenic Aß oligomers, while leaving patient-derived samples containing only inert monomer unchanged. (b) Picomolar concentrations of AP oligomers were incubated with excess AP monomer (O+M), and a sandwich ELISA was preformed to observe an increase in oligomer concentration following incubation. Higher absorbance indicates the presence of greater quantities of oligomer. Oligomer incubated alone (O) and monomer incubated alone (M) serve as comparative controls. Monomer incubated alone exhibits a baseline signal, while incubation of monomer with oligomer increases the absorbance, indicating an increased oligomer content.

The current disclosure shows Aß aggregates induced an increase in permeability and movement of tight junction proteins away from cell borders. Moreover, this response was most pronounced for small aggregates. The smallest Aß aggregates, oligomers, induced a measurable decrease in TEER. This work validates that introduction of physiologically active Aß oligomers can selectively elicit a sensor-based signal. This data provides the basis for the design of a cell-based biosensor for early AD diagnosis that leverages selective BBB breakdown.

The current disclosure provides a means for affordable, early diagnosis of AD. Currently, there is no biomarker-based test to provide definitive diagnosis at early stages of AD. Current diagnostics rely on expensive imaging techniques that detect brain Aß deposition or associated brain atrophy, events that occur at later disease stages. Evidence has shown that an early diagnosis would lead to more effective treatments in part as a result of the irreversible death of neurons.

The current disclosure has demonstrated that introduction of small, physiologically active Aß aggregates, including oligomers, can cause an increase in the permeability of a monolayer of HBMVECs, lowering TEER values. In contrast, non-pathogenetic Aß monomer and mature Aß fibril fail to induce this response. While human BMVEC's are specified, the current disclosure is not so limited. Indeed, other brain cells from a wide host of animals, including mammal, bird, reptile, amphibian and fish, may be utilized. This selective response allows for detection of small quantities of the early, pathogenic form of Aß, even in the presence of larger quantities of inert monomer. A cell-based biosensor will leverage this selectivity to detect the presence of Aß oligomers. This cell-based biosensor will be composed of a monolayer of HBMVECs that when intact adopt tight junctions and provide an high electrical resistance corresponding to BBB cell models. Compromise of this monolayer by physiologically active forms of Aß reduces electrical resistance, with decreases in TEER ranging from 10% to 100%.

Thus, this cell-based biosensor can be exposed to patient cerebral spinal fluid or blood samples that contain physiologically active Aß to render the reduced electrical resistance. In contrast, if only physiologically inert Aß monomer is present in the biological sample, no change in electrical resistance will be observed. If needed, low concentrations of physiologically active Aß oligomers ranging from 0.1 pM to 1 μM can also be amplified via addition of synthetic monomer to increase compromise of the monolayer and induce a larger change in electrical resistance. Oligomer formation by synthetic monomer will be induced by the presence of patient-derived oligomers, but will not occur in the absence of oligomers. Together, the cell-based biosensor design and selective amplification will provide a low-cost means for selectively detecting the presence of physiologically active Aß to diagnose AD at an early stage.

The current disclosure has demonstrated the ability of small Aß aggregates (including oligomers), but not Aß monomer nor Aß fibril, to compromise the integrity of HBMVEC monolayers, leading to increased Pₑ, decreased TEER, migration of tight junction proteins away from cell borders, and activation of the NF-κB transcription factor, which mediates this response. The latter response was observed in the presence of 100 pM Aß, indicating the ability of physiological levels of Aß aggregate concentrations as low as 100 pM to elicit a relevant response. The former response we demonstrated to be specific for the smallest aggregate sizes, including Aß oligomers. Current work involves confirming these responses with biological samples and implementing amplification to bolster measurements of biological samples.

The current disclosure provides for the development of cost-effective, early AD diagnosis. This could be used by physicians in a medical setting who are testing for the disease and has the potential to be marketed to all aging patients as a routine test. In addition, this disclosure could be used in animal studies or clinical studies aimed at developing therapeutics for AD, both to identify candidate subjects and to monitor patient progress.

Various modifications and variations of the described methods, pharmaceutical compositions, and kits of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

What is claimed is:

1. A method for an early detection method of Alzheimer's disease comprising:
measuring transendothelial electrical resistance in a monolayer of brain microvascular endothelial cells;
introducing at least one fluid sample to the monolayer of brain microvascular endothelial cells;
determining if introduction of the at least one fluid sample causes a change in the transendothelial electrical resistance of the monolayer of brain microvascular endothelial cells, wherein a decrease in transendothelial electrical resistance is indicative of Alzheimer's disease.

2. The method of claim 1, wherein the decrease in transendothelial electrical resistance is indicative of the presence of at least one pathogenic Aß aggregate.

3. The method of claim 2, wherein a synthetic monomer is used to amplify a concentration of at least one physiologically active Aß aggregate to increase compromise of the monolayer of brain microvascular endothelial cells.

4. The method of claim 3, wherein no amplification signifies an absence of the at least one pathogenic Aß aggregate.

5. The method of claim 1, wherein the at least one fluid sample comprises at least one cerebral spinal fluid sample and/or at least one blood sample.

6. The method of claim 1, wherein AP aggregation including AP oligomerization is detected.

7. The method of claim 1 further comprising measuring transendothelial transfer rate of the monolayer of brain microvascular endothelial cells.

8. The method of claim 7, wherein the brain microvascular endothelial cells comprise human brain microvascular endothelial cells.

9. The method of claim 7, wherein no change in resistance occurs when at least one Aß monomer or at least one mature Aß fibril is present without presence of at least one pathogenic Aß aggregate.

10. A method for using a cell-based biosensor to detect Alzheimer's Disease comprising:
forming a monolayer of brain microvascular endothelial cells;
exposing the monolayer of brain microvascular endothelial cells to at least one fluid sample; and
determining the presence of at least one pathogenic Aß aggregate in the at least one fluid sample via a change in electrical resistance within the monolayer of brain microvascular endothelial cells, wherein no change in electrical resistance signifies an absence of the at least one pathogenic Aß aggregate.

11. The method of claim 10, wherein a synthetic monomer is used to amplify a concentration of at least one physiologically active Aß aggregate to increase compromise of the monolayer of brain microvascular endothelial cells.

12. The method of claim 11, wherein no amplification signifies an absence of the at least one pathogenic Aß aggregate.

13. The method of claim 10, wherein the fluid samples comprises at least one cerebral spinal fluid sample and/or at least one blood sample.

14. The method of claim 10, wherein Aß aggregation including AP oligomerization is detected.

15. The method of claim 10 further comprising measuring transendothelial transfer rate of the monolayer of brain microvascular endothelial cells.

16. The method of claim 10, wherein the brain microvascular endothelial cells comprise human brain microvascular endothelial cells.

17. The method of claim 10, wherein no change in resistance occurs when at least one Aß monomer or at least one mature Aß fibril is present without presence of at least one pathogenic Aß aggregate.
